(19) United States
(12) Patent Application Publication
(10) Pub. No.: US 2022/0316996 A1
(43) Pub. Date: Oct. 6, 2022

(54) RUTHENIUM OXIDE GAS ABSORBENT LIQUID, ANALYSIS METHOD FOR RUTHENIUM OXIDE, TRAP DEVICE, AND QUANTITATIVE ANALYZER

(71) Applicant: TOKUYAMA CORPORATION, Yamaguchi (JP)

(72) Inventors: Tomoaki SATO, Yamaguchi (JP); Yuki KIKKAWA, Yamaguchi (JP); Takafumi SHIMODA, Yamaguchi (JP); Takayuki NEGISHI, Yamaguchi (JP); Shigenori MAEDA, Yamaguchi (JP)

(73) Assignee: TOKUYAMA CORPORATION, Yamaguchi (JP)

(21) Appl. No.: 17/619,310

(22) PCT Filed: Jun. 17, 2020
(86) PCT No.: PCT/JP2020/023732

§ 371 Date: Dec. 15, 2021

(30) Foreign Application Priority Data
Jun. 21, 2019 (JP) 2019-115740

(51) Int. Cl. G01N 1/22; B01D 53/14; G01N 33/00

(52) U.S. Cl. CPC G01N 1/2214; B01D 53/1493; G01N 33/0027; G01N 2001/2217; B01D 2252/2053

(57) ABSTRACT

A ruthenium oxide gas absorbent liquid includes an organic alkali solution containing a ligand and/or an onium salt composed of an onium ion and an anion, at least part of which is a hydroxide ion, wherein the hydroxide ion has a concentration ranging from more than 1×10⁻⁷ mol/L to 6 mol/L or less.

### TECHNICAL FIELD

The present invention relates to a novel ruthenium oxide gas absorbent liquid that makes it possible to increase the accuracy of analyzing a ruthenium-containing gas generated when a ruthenium-containing semiconductor wafer is processed in a semiconductor element manufacturing process, an analysis method using the absorbent liquid, a trap device, and a quantitative analyzer.

### BACKGROUND ART

In recent years, design rules of semiconductor elements have been miniaturized, and wiring resistance tends to increase. The increase in wiring resistance results in marked hindrance of high-speed operation of the semiconductor element. Thus, some measures are required. Here, it has been desired to provide, as the wiring material, a wiring material having less electromigration resistance and resistivity than conventional wiring materials.

Ruthenium has higher electromigration resistance than conventional wiring materials such as aluminum or copper, and can be used to reduce the resistivity of wiring. Because of this, ruthenium has attracted attention as a wiring material for which the semiconductor element design rule is set to 10 nm or less. In addition to the wiring material, ruthenium can prevent electromigration even when copper is used for the wiring material. Thus, use of ruthenium as a barrier metal for copper wiring has also been considered.

By the way, ruthenium may be selected as a wiring material in a semiconductor element wiring formation process. In this case, dry or wet etching may be used to form wiring like in the case of the conventional wiring materials.

However, in the case of processing by either method, ruthenium transforms to highly volatile RuO₄ (Ru valency is +8; hereinafter, referred to as ruthenium oxide), and part thereof is gasified and released into a gas phase.

Since RuO₄ has strong oxidizing performance, it is harmful to human bodies and there is a concern about health hazards. In addition to this, the oxidizing power of RuO₄ is very strong. Thus, RuO₄ easily reacts with, for instance, an organic material, the surface of a container, or water vapor in the air and readily transforms to RuO₂ (Ru valency is +4), thereby generating RuO₂ particles.

Since the RuO₂ particles accumulate in semiconductor manufacturing equipment (for example, the inside of a chamber, a wafer fixing jig, an exhaust facility) by etching or MP processing, periodical washing and/or replacement work is necessary, causing a decrease in product yield.

The amount of ruthenium oxide generated is preferably smaller and most preferably zero, therefore, etching agents and CMP slurries that are less likely to generate ruthenium oxide gas are being developed.

Accordingly, in order to improve safety and product yield, it is desired to develop an analytical method for quantifying the amount of ruthenium oxide gas generated.

For example, a ruthenium oxide quantification method using a Ru radioisotope contained in ruthenium oxide gas is known in Non-Patent Document 1.

In addition, Patent Document 1 discloses an example in which a semiconductor manufacturing apparatus (dry etching device) and a ruthenium oxide gas concentration measuring instrument are combined, and a gas detecting method using N,N-diethyl-p-phenylenediamine as a color former in combination with an antioxidant and a moisture-proof agent.

### CITATION LIST

### Non Patent Document

Non Patent Document 1: Journal of Nuclear Science and Technology, 1986, Vol. 28, No. 6, p 493-500 Patent Document
Patent Document 1: JP 2002-267606 A

### SUMMARY OF INVENTION

### Technical Problem

However, according to the study of the present inventors, it has been found that the conventional analysis method described in the Citation List has room for improvement in the following points.

For example, the method of Non-Patent Document 1 has high sensitivity, but it is not easy to carry out because it evaluates radio activated ruthenium. Also, in Patent Document 1, a phenomenon where the dye is oxidized by ruthenium oxide to develop a color is utilized, but only coloring is checked. Consequently, the problem is that even coloring caused by an oxidizer other than ruthenium oxide is detected as coloring by ruthenium oxide. Further, if the atmosphere contains water vapor, the error will be large. Thus, it is not particularly suitable for the quantification of ruthenium oxide generated from wet etching or CMP slurry. Moreover, there is a problem that the lower limit of detection is high.

Therefore, there has been a need for a ruthenium oxide quantification method with a low lower limit of quantification applicable to any processing conditions by a simple procedure.

### Solution to Problem

Here, the present inventors have conducted research to solve the above problems by using, as a quantitative method without radio activation, a RuO₄ analysis method using a gas trap liquid.

There are three types of generally known gas trap liquid: acidic one (hydrochloric acid), alkaline one (sodium hydroxide aqueous solution), and low-polarity solvent (carbon tetrachloride). The dissolved state of dissolved ruthenium oxide transforms depending on the properties (pH, polarity) of the gas trap liquid. Ruthenium oxide is said to exist as [RuCl₆]²⁻ or [RuCl₅]²⁻ in hydrochloric acid, RuO₄²⁻ or RuO₄⁻ in NaOH, and RuO₄ in carbon tetrachloride.

Then, the present inventors have studied and found that the dissolved state of ruthenium oxide in the gas trap liquid affects (1) efficiency of trapping a ruthenium oxide gas and (2) efficiency of excitation by inductively coupled plasma (ICP).

As a result of further studies, they have considered that stable trapping and quantification may be possible by trapping ruthenium oxide with sodium hydroxide, converting the ruthenium oxide into a ruthenium chloride complex ion under hydrochloric acid acidic conditions, and then performing measurement by ICP-mediated excitation.

However, when NaOH is used as the gas trap liquid, a large amount of sodium is present in the gas trap liquid.

A problem that high-sensitivity ICP-MS cannot be used occurs newly due to interference by Na. As a countermeasure against this, ICP-OES, which is not interfered by Na, has to be used. Besides, the problem of poor sensitivity, that is, a high lower limit of quantification still remains.

Then, the present inventors have found that use of an organic alkali such as tetramethylammonium hydroxide (TMAH) instead of NaOH makes it possible to utilize ICP-MS and achieve high sensitivity, thereby, the present invention has been completed.

That is, the present invention is configured as follows.

[1] A ruthenium oxide gas absorbent liquid including an organic alkali solution containing an onium salt composed of an onium ion and an anion, at least part of which is a hydroxide ion, wherein the hydroxide ion has a concentration ranging from more than 1×10⁻⁷ mol/L to 6 mol/L or less.
[2] The ruthenium oxide gas absorbent liquid according to [1], wherein the onium ion is a quaternary onium ion represented by formula (1) or a tertiary onium ion represented by formula (2):

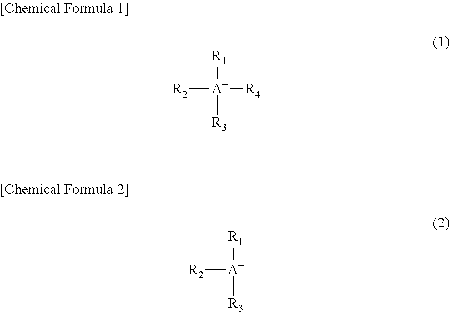

wherein in formula (1), A⁺ is an ammonium ion or a phosphonium ion, and R₁, R₂, R₃, and R₄ are each a C₁₋₂₅ alkyl group, an allyl group, a C₁₋₂₅ alkyl-containing aralkyl group, or an aryl group, provided that when R₁, R₂, R₃, or R₄ is an alkyl group or an allyl group, at least one hydrogen of R₁, R₂, R₃, or R₄ is optionally substituted by a hydroxyl group, a carboxyl group, a cyano group, an amino group, a thiol group, a halogen group, or a sulfonic acid group and at least one hydrogen of a ring of the aryl group or an aryl group in the aralkyl group is optionally substituted by a fluorine atom, a chlorine atom, a C₁₋₁₀ alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom; and in formula (2), A⁺ is a sulfonium ion, and R₁, R₂, and R₃ are each a C₁₋₂₅ alkyl group, an allyl group, a C₁₋₂₅ alkyl-containing aralkyl group, or an aryl group, and at least one hydrogen of a ring of the aryl group or an aryl group in the aralkyl group is optionally substituted by a fluorine atom, a chlorine atom, a C₁₋₁₀ alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom.

[3] The ruthenium oxide gas absorbent liquid according to [2], wherein the onium ion is represented by formula (1), and is a quaternary onium ion where R₁, R₂, R₃, and R₄ are each a C₁₋₄ alkyl group.
[4] The ruthenium oxide gas absorbent liquid according to [2] or [3], wherein the quaternary onium ion is an ammonium ion.
[5] The ruthenium oxide gas absorbent liquid according to [4], wherein the ammonium ion is a tetraalkylammonium ion.
[6] The ruthenium oxide gas absorbent liquid according to [5], wherein the tetraalkylammonium ion is at least one tetraalkylammonium ion selected from a tetramethylammonium ion, a tetraethylammonium ion, a tetrapropylammonium ion, or a tetrabutylammonium ion.
[7] The ruthenium oxide gas absorbent liquid according to [1], further including, as a ruthenium oxide gas generation inhibitor, an onium salt composed of an onium ion and another anion other than a hydroxide.
[8] The ruthenium oxide gas absorbent liquid according to [7], wherein the other anion is at least one ion selected from a fluoride ion, a chloride ion, an iodide ion, a nitrate ion, a phosphate ion, a sulfate ion, a hydrogen sulfate ion, a methane sulfate ion, a perchlorate ion, a chlorate ion, a chlorite ion, a hypochlorite ion, an orthoperiodate ion, a metaperiodate ion, an iodate ion, an iodite ion, a hypoiodite ion, an acetate ion, a carbonate ion, a bicarbonate ion, a fluoroborate ion, or a trifluoroacetate ion.
[9] The ruthenium oxide gas absorbent liquid according to [1], further including, as a ruthenium oxide gas generation inhibitor, a ligand coordinated to ruthenium.
[10] The ruthenium oxide gas absorbent liquid according to [9], wherein the ligand coordinated to ruthenium is oxalic acid, dimethyl oxalate, 1,2,3,4,5,6-cyclohexanecarboxylic acid, succinic acid, acetic acid, butane-1,2,3,4-tetracarboxylic acid, dimethylmalonic acid, glutaric acid, di-glycolic acid, citric acid, malonic acid, 1,3-adamantane dicarboxylic acid, or 2,2-bis(hydroxymethyl)propionic acid.
[11] The ruthenium oxide gas absorbent liquid according to any one of [1] to [10], wherein the onium ion has a concentration of from 1×10⁻³ mol/L to 8 mol/L.
[12] The ruthenium oxide gas absorbent liquid according to any one of [1] to [11], further including water or an organic solvent.
[13] The ruthenium oxide gas absorbent liquid according to [12], wherein the organic solvent has a relative permittivity of 45 or less.
[14] The ruthenium oxide gas absorbent liquid according to [12] or [13], wherein the organic solvent is at least one compound selected from the group consisting of sulfolanes, alkylnitriles, halogenated alkanes, ethers, esters, aldehydes, ketones, and alcohols.
[15] The ruthenium oxide gas absorbent liquid according to any one of [12] to [14], wherein the organic solvent in a treatment liquid has a concentration of 0.1 mass % or more.
[16] An analysis method for ruthenium oxide in a process gas, the method including: bringing a ruthenium oxide gas-containing process gas into contact with the ruthenium oxide gas absorbent liquid according to any one of [1] to [15] to recover the ruthenium oxide gas from the process gas; and then analyzing an amount of ruthenium oxide in the ruthenium oxide gas absorbent liquid.
[17] The analysis method according to [16], wherein the process gas is derived from a semiconductor-use chemical liquid used for processing a ruthenium metal-containing semiconductor material.
[18] A trap device including a trap unit disposed in an exhaust path at a step of processing a ruthenium metal-containing semiconductor device and configured to recover a ruthenium oxide component in an exhaust gas, the trap unit including: as a means for trapping ruthenium oxide contained in the exhaust gas, a container filled with the ruthenium oxide gas absorbent liquid according to any one of [1] to [17]; a supply means; and a drainage pipe for discharging the ruthenium oxide gas absorbent liquid after absorption.
[19] A ruthenium oxide gas quantitative analyzer including:

a trap device including a trap unit disposed in an exhaust path at a step of processing a ruthenium metal-containing semiconductor device and configured to recover a ruthenium oxide component in an exhaust gas, the trap unit including, as a means for trapping ruthenium oxide contained in the exhaust gas, a container filled with the ruthenium oxide gas absorbent liquid according to any one of [1] to [17], a supply means, and a drainage pipe for discharging the ruthenium oxide gas absorbent liquid after absorption; and

an analysis means for quantifying the ruthenium oxide component in the absorbent liquid sampled from the trap device.

[20] The quantitative analyzer according to [19], wherein the analysis means is an analysis means using ICP emission spectrometry, ICP mass spectrometry, or ultraviolet-visible spectroscopy (UV-VIS).

### Advantageous Effects of Invention

The present invention makes it possible to, by using a solution containing a metal-free organic alkali as a ruthenium oxide gas absorbent liquid, quantify ruthenium oxide with high sensitivity by ICP-MS.

In addition, since the ruthenium oxide gas absorbent liquid contains a given ruthenium oxide gas generation inhibitor, the ruthenium oxide trapping efficiency is increased, the volume of the ruthenium oxide gas absorbent liquid can be reduced, and the sensitivity of quantification can be increased without performing an operation such as concentration of the absorbent liquid. This is effective not only for highly sensitive ICP-MS but also for ICP-OES.

Further, when a ruthenium oxide gas generation inhibitor is included in the ruthenium oxide gas absorbent liquid, each cation contained in the inhibitor forms an ion pair with RuO₄²⁻ and/or RuO₄⁻ in the absorbent liquid, so that ruthenium oxide can be efficiently collected.

Furthermore, the present invention also provides an analysis method, a trap device, and an analyzer such that this ruthenium oxide gas absorbent liquid can be used to analyze efficiently, simply, and accurately ruthenium oxide in a process gas generated during a semiconductor processing process.

### BRIEF DESCRIPTION OF DRAWINGS

FIG. 1 shows a schematic view of a device evaluated in Examples and Comparative Example.

### DESCRIPTION OF EMBODIMENTS

Hereinafter, embodiments of the present invention will be described. However, the present invention is no way limited to the description.

### (Ruthenium Oxide Gas Absorbent Liquid)

A ruthenium oxide gas absorbent liquid of the present invention includes an organic alkali containing an onium salt composed of an onium ion and an anion, at least part of which is a hydroxide ion. Use of an organic alkali makes it possible to perform highly sensitive analysis of ruthenium oxide by ICP-MS. ICP-MS has a potential allowing for highly sensitive analysis of ruthenium oxide. Meanwhile, a liquid containing an inorganic alkali (for example, NaOH, KOH) may be used as a gas absorbent liquid. In this case, Na and/or K can cause interference. Thus, it is difficult to analyze a tiny amount of ruthenium oxide. Then, instead of the inorganic alkali, an organic alkali such as tetramethylammonium hydroxide (TMAH) may be used. By doing so, it is possible to eliminate the interference caused by Na and/or others. This enables highly sensitive analysis of ruthenium oxide by ICP-MS.

The onium ion is a quaternary onium ion, a tertiary onium ion, a secondary onium ion, or an onium ion substituted with hydrogen. For example, the onium ion is a cation such as an ammonium ion, a phosphonium ion, a fluoronium ion, a chloronium ion, a bromonium ion, an iodonium ion, an oxonium ion, a sulfonium ion, a selenonium ion, a telluronium ion, an arsonium ion, a stibonium ion, or a bismutonium ion.

In the present invention, the onium ion is preferably a quaternary onium ion represented by formula (1) or a tertiary onium ion represented by formula (2).

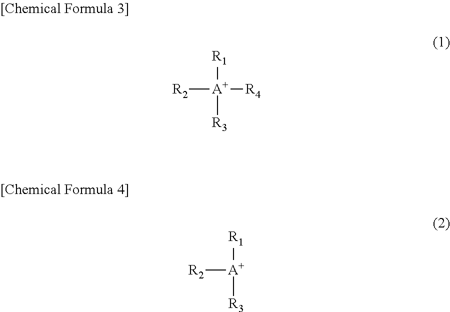

In the formula (1), A⁺ represents an ammonium ion or a phosphonium ion.

R₁, R₂, R₃, and R₄ are each a C₁₋₂₅ alkyl group, an allyl group, a C₁₋₂₅ alkyl-containing aralkyl group, or an aryl group. Provided that when R₁, R₂, R₃, or R₄ is an alkyl group or an allyl group, at least one hydrogen of R₁, R₂, R₃, or R₄ is optionally substituted by a hydroxyl group, a carboxyl group, a cyano group, an amino group, a thiol group, a halogen group, or a sulfonic acid group. In addition, when R₁, R₂, R₃, or R₄ is an alkyl group or an allyl group, R₁, R₂, R₃, or R₄ optionally contains a carbonyl group. Further, at least one hydrogen of an aryl group or a ring of the aryl group in the aralkyl group is optionally substituted by a fluorine atom, a chlorine atom, a C₁₋₁₀ alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom.

In formula (2), A⁺ is a sulfonium ion, and R₁, R₂, and R₃ are each a C₁₋₂₅ alkyl group, an allyl group, a C₁₋₂₅ alkyl-containing aralkyl group, or an aryl group. In addition, at least one hydrogen of an aryl group or a ring of the aryl group in the aralkyl group is optionally substituted by a fluorine atom, a chlorine atom, a C₁-10 alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom.

Since the ruthenium oxide gas absorbent liquid of the present invention is alkaline, the absorbed ruthenium oxide exists as an anion such as RuO₄⁻ or RuO₄²⁻ (hereinafter, sometimes referred to as RuO₄⁻, for example). RuO₄⁻, for example, can form an ion pair by electrostatically interacting with an onium ion in the ruthenium oxide gas absorbent liquid and can thus stably exist in the ruthenium oxide gas absorbent liquid. Because of this, the ruthenium oxide gas absorbent liquid containing an onium ion should efficiently absorb the ruthenium oxide gas.

The alkyl group of R₁, R₂, R₃, or R₄ in formula (1) or (2) can be used without particular limitation as long as the alkyl group contains 1 to 25 carbon atoms. As the number of carbon atoms increases, the onium ion more strongly interacts with RuO₄⁻, for example, so that RuO₄ can be efficiently collected. On the other hand, as the number of carbon atoms increases, the onium ion becomes bulkier. This causes a decrease in the solubility of the ion pair with RuO₄⁻, for example, in the ruthenium oxide gas absorbent liquid, thereby causing a precipitate. Since this precipitate affects excitation by ICP, it is preferable that the precipitate should not occur. By contrast, when the number of carbon atoms is small, the interaction between the onium ion and RuO₄⁻, for example, is weakened and RuO₄ gas trapping effect is reduced, but, the handling is easy because foaming in the ruthenium oxide gas absorbent liquid is suppressed.

Hence, in an onium salt of the organic alkali, the number of carbon atoms in the alkyl group of formula (1) or (2) is preferably from 1 to 25, more preferably from 1 to 10, and most preferably from 1 to 4. When an onium salt has an alkyl group such a carbon number, RuO₄ gas can be collected in the absorbent liquid by interaction with RuO₄⁻, for example, and a precipitate is hardly generated, therefore, the onium salt can be suitably used in the absorbent liquid

The aryl group of R₁, R₂, R₃, or R₄ in formula (1) or (2) includes not only an aromatic hydrocarbon but also a heteroaryl containing a heteroatom, and is not particularly limited. Here, a phenyl group or a naphthyl group is preferable.

The quaternary onium ion is preferably an ammonium ion or a phosphonium ion that can be stably present in a treatment liquid. In general, the alkyl chain length of an ammonium ion or a phosphonium ion can be easily controlled, further, an allyl group or an aryl group can be easily introduced. This makes it possible to control, for instance, the size, symmetry, hydrophilicity, hydrophobicity, stability, solubility, charge density, and interface activity performance of the ammonium ion or phosphonium ion. Since such an ammonium ion or phosphonium ion easily forms an ion pair with RuO₄⁻, for example, in the ruthenium oxide gas absorbent liquid, the ruthenium oxide gas absorbent liquid containing the onium salt can efficiently trap the ruthenium oxide gas.

The tertiary onium ion is preferably a sulfonium ion that can be stably present in a treatment liquid. In general, the alkyl chain length of a sulfonium ion can be easily controlled, and an allyl group or an aryl group can be easily introduced. This makes it possible to control, for instance, the size, symmetry, hydrophilicity, hydrophobicity, stability, solubility, charge density, and interface activity performance of the sulfonium ion. Since such a sulfonium ion easily forms an ion pair with RuO₄⁻, for example, in the ruthenium oxide gas absorbent liquid, the ruthenium oxide gas absorbent liquid containing the onium salt can efficiently trap the ruthenium oxide gas.

R₁, R₂, R₃, and R₄ in formula (1) or (2) may be the same group or different groups, but are preferably the same group in view of industrial availability, stability, and others.

In the present invention, the onium ion is preferably a quaternary onium ion represented by formula (1) where R₁, R₂, R₃, and R₄ are each a C₁₋₄ alkyl group, and more preferably an ammonium ion because of high stability, industrial availability of high purity products, and low cost. The ammonium ion is preferably a tetraalkylammonium ion, and the tetraalkylammonium ion is preferably at least one ammonium ion selected from a tetramethylammonium ion, a tetraethylammonium ion, a tetrapropylammonium ion, or a tetrabutylammonium ion.

The onium ion to be added for trapping RuO₄⁻, for example, in the gas absorbent liquid may be those that cause electrostatic interaction with RuO₄⁻, for example, as described above. Due to this, the structure of the onium ion is not limited to the quaternary onium ion represented by the above-described formula (1) or the tertiary onium ion represented by formula (2). Specifically, an onium salt different from that in the organic alkali containing the onium ion represented by formula (1) or (2) may include, for instance, an onium ion having a cyclic structure (for example, an imidazolium ion, a pyrrolidinium ion, a pyridinium ion, a piperidinium ion, or an oxazolium ion) or a dication represented by a hexamethonium ion.

Each onium ion is used in combination with an anion to form an onium salt, which is added to the ruthenium oxide gas absorbent liquid. This onium salt traps an ion (RuO₄⁻, for example) containing a ruthenium atom generated by ruthenium dissolution, thereby contributing to an increase in the ruthenium oxide trapping efficiency.

The organic alkali composed of the above onium ion and a hydroxide ion is contained in the absorbent liquid so that the hydroxide ion has a concentration of more than 1×10⁻⁷ mol/L and 6 mol/L or less. The upper limit of the concentration is preferably 5 mol/L and more preferably 4 mol/L. This concentration makes the viscosity of the absorbent liquid lower and the handling easier. Still more preferably, the upper limit of the concentration is 2.8 mol/L. When this upper limit is said concentration, the organic alkali having high stability and high purity is industrially available at low cost. The lower limit of the concentration is preferably 1×10⁻⁴ mol/L and more preferably 1×10⁻³ mol/L. This lower limit can make ruthenium oxide stable as RuO₄⁻ or RuO₄²⁻ in the absorbent liquid, so that the trapping efficiency in the absorbent liquid can be increased.

The concentration of the onium ion is from 1×10⁻⁷ mol/L to 8 mol/L, and preferably from 1×10⁻³ mol/L to 8 mol/L. A certain amount of the onium ion is required to efficiently trap the ruthenium oxide gas. However, if the concentration is too high, the onium salt is not dissolved, and an ion pair of the onium ion and RuO₄⁻, for example, may precipitate.

The onium ion concentration in the absorbent liquid is the total concentration of the onium ion derived from the organic alkali and the onium ion(s) derived from the onium salt(s) other than the organic alkali.

### (Ruthenium Oxide Gas Generation Inhibitor)

To adjust the onium ion concentration within the above range, an onium salt(s) other than the hydroxide may be added to the ruthenium oxide gas absorbent liquid. Such an onium salt other than the hydroxide is referred to as a ruthenium oxide gas generation inhibitor. In view of the onium ion concentration and the hydroxide ion concentration, the concentration of the onium salt(s) other than the hydroxide is desirably in the range of from 0.0001 to 3 mol/L.

The onium salt added as the ruthenium oxide gas generation inhibitor is composed of an onium ion and an anion, and this anion is different from that of the onium hydroxide salt constituting the organic alkali. Examples of the onium ion include the same ones as those described above. Note that the onium ion constituting the onium salt as the ruthenium oxide gas generation inhibitor may be the same as or different from the onium ion constituting the organic alkali.

Here, in the onium salt of the ruthenium oxide gas generation inhibitor, the number of carbon atoms of the alkyl group in formula (1) or (2) is preferably from 1 to 25, more preferably from 2 to 10, and most preferably from 3 to 6.

This anion is not particularly limited as long as it is other than a hydroxide ion. Specifically, the anion is, preferably, at least one kind selected from a fluoride ion, a chloride ion, an iodide ion, a nitrate ion, a phosphate ion, a sulfate ion, a hydrogen sulfate ion, a methane sulfate ion, a perchlorate ion, a chlorate ion, a chlorite ion, a hypochlorite ion, an orthoperiodate ion, a metaperiodate ion, an iodate ion, an iodite ion, a hypoiodite ion, an acetate ion, a carbonate ion, a bicarbonate ion, a fluoroborate ion, or a trifluoroacetate ion.

In addition, as the ruthenium oxide gas generation inhibitor, a ligand having an ability to coordinate to ruthenium may be added.

### (Ligand Having Ability to Coordinate to Ruthenium)

In addition, the ruthenium oxide trapping efficiency can also be improved by adding, to the ruthenium oxide gas absorbent liquid, a ligand having an ability to coordinate to ruthenium (hereinafter, sometimes referred to as a “ligand”). Examples of such a ligand include a compound having, for instance, an amino group, a phosphino group, a carboxyl group, a carbonyl group, or a thiol group containing, for example, a nitrogen, phosphorus, oxygen, or sulfur atom(s). Of course, the ligand is not limited to them. Due to the difference in electronegativity between ruthenium and oxygen in ruthenium oxide, ruthenium exists in a state where the charge is shifted to the positive side. When the lone electron pair contained in the ligand is coordinated to this ruthenium, ruthenium oxide can exist more stably in the ruthenium oxide gas absorbent liquid. This seems to increase the ruthenium oxide trapping efficiency.

In addition, in a ligand having a C═O bond, for example, a carbonyl group or a carboxyl group, the following gas suppression mechanism also occurs, so that the ruthenium oxide trapping efficiency is increased. Ruthenium oxide is generally known as a metal oxide having strong electrophilicity because ruthenium constituting the ruthenium oxide has high electronegativity among metals. Since a highly electrophilic metal oxide is easily coordinated to an unsaturated bond carbon, ruthenium is coordinated to a compound containing a carbonyl group having an unsaturated bond. The ruthenium oxide trapping efficiency seems to be increased because a chemical species, in which such a carbonyl group-containing compound and ruthenium oxide are interacted, is stably present in the ruthenium oxide gas absorbent liquid. As the carbonyl group-containing compound, particularly preferred is a ketone, a carboxylic acid, an ester, an amide, an enone, an acid chloride, or an acid anhydride that is highly stable to an oxidizer.

As described above, a case where the ligand is coordinated to ruthenium or a case where the ligand and ruthenium are subject to back coordination is considered. In the present application, either case falls under the ligand having an ability to coordinate to ruthenium. The ligand having an ability to coordinate to ruthenium may be added alone to the ruthenium oxide gas absorbent liquid, or may be used in combination with an onium salt as the above-described ruthenium oxide gas generation inhibitor.

If the ligand contains a hydrocarbon group, the number of carbon atoms in the hydrocarbon group is preferably 10 or less in order to maintain the solubility necessary for effective trapping. When a plurality of hydrocarbon groups are contained, the number of carbon atoms in each hydrocarbon group is preferably 10 or less. Since a larger number of carbon atoms causes an increase in the molecular weight, this results in a decrease in the solubility of the ligand in the absorbent liquid. Since the decrease in the solubility causes a decrease in the concentration of the ligand present in the absorbent liquid, this results in a decrease in the effect of trapping ruthenium oxide gas.

As described in the mechanism of trapping ruthenium oxide gas by the ligand, since 0 having a lone electron pair is coordinated to ruthenium in ruthenium oxide, the ligand preferably contains a hydroxyl group and/or an ether bond.

Based on the reasons described above, the ligand that can be preferably used in the present invention is as follows.

Preferable examples include: an amine compound (for example, triethanolamine, nitrilotriacetic acid, ethylenediaminetetraacetic acid, glycine, phthalic acid); a thiol compound (for example, cysteine, methionine); a phosphine compound (for example, tributylphosphine, tetramethylenebis(diphenylphosphine)); a monocarboxylic acid (for example, acetic acid, formic acid, lactic acid, glycolic acid, 2,2-bis(hydroxymethyl)propionic acid, gluconic acid, α-glucoheptoic acid, heptinic acid, phenylacetic acid, phenylglycolic acid, benzylic acid, gallic acid, cinnamic acid, naphthoic acid, anisic acid, salicylic acid, cresotic acid, acrylic acid, benzoic acid); a dicarboxylic acid (for example, malic acid, adipic acid, succinic acid, maleic acid, tartaric acid, oxalic acid, dimethyl oxalate, glutaric acid, malonic acid, 1,3-adamantandicarboxylic acid, diglycolic acid); a tricarboxylic acid (for example, citric acid); a tetracarboxylic acid represented by butane-1,2,3,4-tetracarboxylic acid; a hexacarboxylic acid represented by 1,2,3,4,5,6-cyclohexanehexacarboxylic acid; or a carbonyl compound (for example, ethyl acetoacetate, dimethylmalonate).

More preferable examples include: a monocarboxylic acid (for example, acetic acid, formic acid, lactic acid, glycolic acid, 2,2-bis(hydroxymethyl)propionic acid, gluconic acid, α-glucoheptoic acid, heptinic acid, phenylacetic acid, phenylglycolic acid, benzylic acid, gallic acid, cinnamic acid, naphthoic acid, anisic acid, salicylic acid, cresotic acid, acrylic acid, benzoic acid); a dicarboxylic acid (for example, malic acid, adipic acid, succinic acid, maleic acid, tartaric acid, oxalic acid, dimethyl oxalate, glutaric acid, malonic acid, 1,3-adamantandicarboxylic acid, diglycolic acid); a tricarboxylic acid (for example, citric acid); a tetracarboxylic acid represented by butane-1,2,3,4-tetracarboxylic acid; a hexacarboxylic acid represented by 1,2,3,4,5,6-cyclohexanehexacarboxylic acid; or a carbonyl compound (for example, ethyl acetoacetate, dimethylmalonate).

Still more preferable examples include oxalic acid, dimethyl oxalate, 1,2,3,4,5,6-cyclohexanecarboxylic acid, succinic acid, acetic acid, butane-1,2,3,4-tetracarboxylic acid, dimethylmalonic acid, glutaric acid, di-glycolic acid, citric acid, malonic acid, 1,3-adamantane dicarboxylic acid, or 2,2-bis(hydroxymethyl)propionic acid.

The concentration of the ligand in the ruthenium oxide gas absorbent liquid is preferably from 0.0001 to 60 mass %. If the amount of the ligand added is too small, not only the interaction with ruthenium oxide is weakened to reduce the effect of trapping the ruthenium oxide gas, but also the amount of ruthenium oxide that can be dissolved in the absorbent liquid is reduced. Thus, the analysis sensitivity is lowered. On the other hand, if the addition amount is too large, a precipitate composed of the ligand and ruthenium oxide is likely to be generated. In addition, interference occurs in ICP-MS, so that quantification becomes difficult. Thus, in the absorbent liquid of the present invention the content of the ligand is preferably from 0.0001 to 60 mass %, more preferably from 0.01 to 35 mass %, and still more preferably from 0.1 to 20 mass %. Note that in the case of adding the ligand, only one kind may be added, or two or more kinds may be added in combination. Two or more kinds of the ligands may be included. Even in the case including two or more kinds of the ligands, if the total concentration of the ligands is within the above concentration range, the ruthenium oxide gas can be effectively collected.

Inclusion of the ligand(s) in the absorbent liquid makes it possible to reduce the volume of the absorbent liquid. Alternatively, in addition to the ligand(s), an onium salt other than the hydroxide is added to the absorbent liquid. This enables the volume of the absorbent liquid to be further reduced. Accordingly, inclusion of the ruthenium oxide gas generation inhibitor makes it possible to lower the lower limit of quantification of ruthenium oxide.

In addition, an additional organic alkali compound other than the onium hydroxide and/or ammonia may be included. The additional organic alkali compound is an alkaline compound having a carbon atom(s), and is preferably (a) a hydrocarbon amine compound containing 3 or more carbon atoms, or (b) an amine compound containing an oxygen atom or a sulfur atom. Here, the amine compound is a compound including a primary amine, a secondary amine, a tertiary amine, or a salt thereof. It is assumed that the examples also include a carbamoyl group or a salt thereof.

Examples of the hydrocarbon group of the hydrocarbon amine compound (a) include an alkane residue (typically an alkyl group, but may be a polyvalent group), an alkene residue, an aryl residue, or a combination thereof. Specific examples thereof include cyclohexylamine, pentylamine, benzylamine, n-hexylamine, 2-ethylhexylamine, or octylamine.

The oxygen atom- or sulfur atom-containing amine compound (b) is preferably a compound having the above-defined hydrocarbon group and a substituent containing an oxygen atom or a sulfur atom. Specific examples thereof include a hydroxy group (OH), a carboxyl group (COOH), a mercapto group [a sulfanyl group (SH)], an ether group (0), a thioether group (S), or a carbonyl group (CO). The amine compound (b) contains one or more carbon atoms.

Specific examples of the oxygen atom- or sulfur atom-containing amine compound (b) include methyl carbazate, O-methylhydroxylamine, N-methylhydroxylamine, monoethanolamine, 3-ethoxypropylamine, diglycolamine, triethanolamine, diethanolamine, monoethanolamine, N-methylethanolamine, N,N-diethylmonoethanolamine, diethylhydroxylamine, isopropanolamine, diisopropanolamine, or 2-(methylamino)ethanol.

The solvent for the ruthenium oxide gas absorbent liquid is usually water. The water contained in the treatment liquid in the present invention is preferably water from which, for example, metal ions, organic impurities, and particle particles have been removed by distillation, ion exchange treatment, filter treatment, various adsorption treatments, and others. Pure water or ultrapure water is particularly preferable. Such water can be obtained by a known procedure widely used in semiconductor manufacturing.

### (Organic Solvent)

As described above, the onium ions present in the ruthenium oxide gas absorbent liquid in the present invention electrostatically interact with RuO₄⁻, for example. The RuO₄⁻, for example, as an ion pair is retained in the absorbent liquid to increase the RuO₄ gas trapping efficiency. In this case, RuO₄⁻, for example, and each onium ion are dissolved in the solution in the form of ion pair. When exceeding the solubility, a precipitate occurs, the precipitate causes an error in the RuO₄ gas quantification. Thus, it is important not to cause the precipitate, and it is preferable to increase the solubility of the ion pair. For this procedure, addition of an organic solvent is effective. When ruthenium oxide and a chemical species consisting of a ligand having an ability to coordinate to the ruthenium oxide form a precipitate, as like in the case of the above-mentioned ion pair composed of the onium ion and RuO₄⁻, an organic solvent may be added to the ruthenium oxide gas absorbent liquid. This makes it possible to increase the solubility of the species and reduce the error in the RuO₄ gas quantification. The following describes the effect of the organic solvent by taking as an example the case where an onium ion present in the ruthenium oxide gas absorbent liquid forms an ion pair with RuO₄⁻, for example. Here, when a chemical species consisting of ruthenium oxide and a ligand is formed, the term “ion pair” in the following description may be read as the chemical species consisting of ruthenium oxide and a ligand.

In general, the lower the relative permittivity of the solvent, the more easily a chemical species that is electrically neutral is dissolved. The ion pair that is electrically neutral is also more easily dissolved as the relative permittivity of the solvent becomes lower. Thus, to increase the solubility of the ion pair, it is desirable to add an organic solvent having a relative permittivity lower than that of water (relative permittivity: 78) as the organic solvent to be added to the ruthenium oxide gas absorbent liquid in the present invention. In this way, since the relative permittivity of the ruthenium oxide gas absorbent liquid can be made lower than in the case of using just water, this can increase the solubility of the ion pair composed of the onium ion and RuO₄⁻, for example, thereby capable of suppressing precipitation of the ion pair. It is possible to add, as the organic solvent to be added, any organic solvent as long as the relative permittivity is lower than that of water. The relative permittivity is preferably 45 or less, more preferably 20 or less, and still more preferably 10 or less. Note that each relative permittivity is a value at 25° C.

Examples of such an organic solvent include an alkylnitrile compound, an aldehyde compound, an ether compound, an ester compound, a ketone compound, a sulfolane compound, a halogenated alkane compound, or an alcohol compound.

More specific examples include 1,4-dioxane (relative permittivity 2.2), carbon tetrachloride (relative permittivity 2.2), benzene (relative permittivity 2.3), toluene (relative permittivity 2.4), propionic acid (relative permittivity 3.4), trichloroethylene (relative permittivity 3.4), diethyl ether (relative permittivity 4.3), chloroform (relative permittivity 4.9), acetic acid (relative permittivity 6.2), methyl benzoate (relative permittivity 6.6), methyl formate (relative permittivity 8.5), phenol (relative permittivity 9.8), p-cresol (relative permittivity 9.9), isobutyl alcohol (relative permittivity 17.9), acetone (relative permittivity 20.7), nitroethane (relative permittivity 28.1), acetonitrile (relative permittivity 37), ethylene glycol (relative permittivity 37.7), or sulfolane (relative permittivity 43). Of course, the organic solvent is not limited to them.

When an organic solvent having a low relative permittivity is added, it may be difficult to mix with water. However, even in such a case, an organic solvent that has been slightly dissolved in water may be used to increase the solubility of the ion pair. Accordingly, the organic solvent can be added to effectively suppress formation of the precipitate.

The organic solvent may be added in an amount necessary for suppressing the formation of the precipitate. To achieve this, the concentration of the organic solvent in the ruthenium oxide gas absorbent liquid may be 0.1 mass % or more. Here, the amount of the ion pair dissolved should be increased to stably retain RuO₄⁻, for example, as an ion pair in the ruthenium oxide gas absorbent liquid. For this purpose, the concentration of the organic solvent is preferably 1 mass % or more. The range may be set so as not to impair the solubilities of the ruthenium oxide gas, RuO₄⁻, for example, and the onium salt and/or the stability of the ruthenium oxide gas absorbent liquid. As the volume of the organic solvent added becomes larger, the amount of the ion pair that can be dissolved in the ruthenium oxide gas absorbent liquid increases, thereby the precipitate formation can be suppressed. In addition, even in the case where a small amount of the organic solvent is evaporated, a decrease in the RuO₄ gas trapping efficiency can be prevented. One kind of the organic solvent may be added, or a plurality of kinds thereof may be added in combination.

Use of a highly volatile solvent as the organic solvent may cause evaporation of the organic solvent in the ruthenium oxide gas absorbent liquid. This may affect the concentration of the organic solvent, thereby affecting the relative permittivity of the ruthenium oxide gas absorbent liquid. As a result, the solubility of the ion pair is modified. Thus, from the viewpoint of measurement stability, a less volatile organic solvent is preferable. Specifically, an organic solvent having a vapor pressure at 20° C. of 50 mmHg or less is preferable, and an organic solvent having a vapor pressure of 20 mmHg or less is more preferable.

### (Additional Additives)

An additional additive(s) may be blended in the ruthenium oxide gas absorbent liquid of the present invention as long as the objective of the present invention is not impaired. Examples of the additional additive that can be added include an acid, a metal anticorrosive, a water-soluble organic solvent, a fluorine compound, an oxidizer, a reductant, a complexing agent, a chelator, a surfactant, an antifoaming agent, a pH modifier, or a stabilizer. These additives may be added singly or in combination.

The following may be derived from any of the above additives and in view of matters upon the treatment liquid production, the treatment liquid in the present invention optionally contains an alkali metal ion (for example, a sodium ion, a potassium ion) or an alkaline earth metal ion (for example, a calcium ion). Thus, for example, the pH modifier may contain an alkali metal hydroxide (for example, sodium hydroxide) or an alkaline earth metal hydroxide. However, the alkali metal ion or alkaline earth metal ion, for instance, may affect the quantitative analysis. Due to this, the smaller amount is better. In practice, the amount should be as little as possible.

Specifically, the total amount of the alkali metal ion and the alkaline earth metal ion is preferably 1% by mass or less, more preferably 0.7% by mass or less, still more preferably 0.3% by mass or less, particularly preferably 10 ppm or less, and most preferably 500 ppb or less.

### (Analysis Method for Ruthenium Oxide)

An analysis method for ruthenium oxide in a process gas according to an embodiment of the present invention includes: bringing a ruthenium oxide gas-containing process gas into contact with the ruthenium oxide gas absorbent liquid described above to recover the ruthenium oxide gas from the process gas; and then analyzing an amount of ruthenium oxide in the ruthenium oxide gas absorbent liquid.

The process gas is a ruthenium oxide-containing gas, and it is preferable to use a gas derived from a semiconductor-use chemical liquid used for processing a ruthenium metal-containing semiconductor material. Examples include a ruthenium oxide gas generated at the time of processing, for instance, a substrate including a semiconductor (for example, Si) having ruthenium wiring thereon. In addition, it is also preferable to use a gas containing a ruthenium oxide gas generated during, for example, an etching step, a residue removing step, a cleaning step, and/or a CMP step in a semiconductor manufacturing process.

The ruthenium source applied to the ruthenium oxide gas absorbent liquid of the present invention may be formed by any method. Ruthenium may be deposited using a widely known method in a semiconductor manufacturing process, such as CVD, ALD, sputtering, or plating. The ruthenium may be metal ruthenium, a ruthenium oxide, an alloy with another metal, an intermetallic compound, an ionic compound, or a complex. In addition, the ruthenium may be exposed on the surface of a wafer, or may be covered with, for example, another metal, a metal oxide film, an insulating film, or a resist. Even if ruthenium is covered with another material, a ruthenium oxide gas is generated when ruthenium is dissolved in the processing step.

For the ruthenium oxide gas absorbent liquid after the ruthenium oxide gas has been recovered, the amount of ruthenium oxide in the gas absorbent liquid may be analyzed by a known analysis method. The analysis method is not particularly limited. For example, as an analysis method for radioactive ruthenium, it is also possible to adopt a method in which ruthenium is separated by magnesium hydroxide co-precipitation-distillation or carbon tetrachloride extraction, and then reduced to precipitate ruthenium for quantification. The amount of ruthenium oxide in the ruthenium oxide gas absorbent liquid can also be quantified by ultraviolet-visible spectroscopy (UV-VIS). Further, atomic absorption spectrometry or inductively coupled plasma emission spectroscopy can also be used as the analysis means. In particular, from the viewpoint of simplicity and accuracy, an analysis means by ICP emission spectrometry (ICP-OES) or ICP mass spectrometry (ICP-MS) is preferable. Since the ruthenium oxide trapping efficiency in the present invention is high, the absorbent liquid does not have to be concentrated. In addition, since an organic alkali is used, Thus, highly sensitive analysis using ICP-MS can be carried out.

The ruthenium oxide gas absorbent liquid of the present invention is prepared by adjusting the type, the hydrocarbon chain length, and the concentration of each of the cation or the anion constituting the organic alkali or the ruthenium oxide gas generation inhibitor. This can balance between the ruthenium oxide trapping efficiency and the detection efficiency. In addition, use of the organic alkali makes it possible to lower the lower limit of quantification of ruthenium oxide gas to about 1/500 to 1/1000 when compared with the case of using an inorganic alkali (for example, NaOH or KOH). Further, a ruthenium oxide gas generation inhibitor may be added to the absorbent liquid to reduce the liquid volume at the time of analysis to about ½ to ⅕. As a result, the sensitivity can be increased by about 2 to 5 folds.

Then, the organic alkali and the ruthenium oxide gas generation inhibitor may be used in combination to lower the lower detection limit by up to about 1/5000.

Furthermore, an absorbent liquid containing an organic alkali containing an onium salt used in the present invention can serve as an alkali and exert both the effect of absorbing a ruthenium oxide gas and the effect of the ruthenium oxide gas generation inhibitor. This makes it suitable as the ruthenium oxide gas absorbent liquid.

A trap device using such an absorbent liquid according to an embodiment includes a trap device including a trap unit disposed in an exhaust path at a step of processing a ruthenium metal-containing semiconductor device and configured to recover a ruthenium oxide component in an exhaust gas,

the trap unit including: as a means for trapping ruthenium oxide contained in the exhaust gas, a container filled with the ruthenium oxide gas absorbent liquid; a supply means; and a drainage pipe for discharging the ruthenium oxide gas absorbent liquid after absorption.

Also, examples of an analyzer using the trap device according to an embodiment include a ruthenium oxide gas quantitative analyzer including: a trap device including a trap unit disposed in an exhaust path at a step of processing a ruthenium metal-containing semiconductor device and configured to recover a ruthenium oxide component in an exhaust gas,

the trap unit including, as a means for trapping ruthenium oxide contained in the exhaust gas, a container filled with the ruthenium oxide gas absorbent liquid, a supply means, and a drainage pipe for discharging the ruthenium oxide gas absorbent liquid after absorption; and an analysis means for quantifying the ruthenium oxide component in the absorbent liquid sampled from the trap device.

As the analysis means, the above-described one can be used, and in particular, an analysis means using ICP-OES, ICP-MS, or UV-VIS is preferable because it is simple and highly accurate.

### EXAMPLES

Hereinafter, the present invention will be specifically described with reference to Examples. However, the present invention is not limited to these Examples.

To Prepare Absorbent Liquid

The following ruthenium oxide gas absorbent liquids 1 to 10 were prepared.

(1) Ruthenium Oxide Gas Absorbent Liquid 1 (Example 1)

An aqueous solution containing tetramethylammonium hydroxide (TMAH), as an organic alkali, at a concentration of 1 mol/L was prepared.

(2) Ruthenium Oxide Gas Absorbent Liquid 2 (Example 2)

An aqueous solution containing tetramethylammonium hydroxide (TMAH), as an organic alkali, at a concentration of 1.0×10⁻³ mol/L was prepared.

(3) Ruthenium Oxide Gas Absorbent Liquid 3 (Example 3)

An aqueous solution containing tetramethylammonium hydroxide (TMAH), as an organic alkali, at a concentration of 2 mol/L was prepared.

(4) Ruthenium Oxide Gas Absorbent Liquid 4 (Example 4)

An aqueous solution containing tetramethylammonium hydroxide (TMAH), as an organic alkali, at a concentration of 1 mol/L was prepared.

(5) Ruthenium Oxide Gas Absorbent Liquid 5 (Example 5)

An aqueous solution containing tetramethylammonium hydroxide, as an organic alkali, at a concentration of 1 mol/L was prepared, and tetrapropylammonium chloride (TPACl) was added at 5% by mass to prepare ruthenium oxide gas absorbent liquid 5.

(6) Ruthenium Oxide Gas Absorbent Liquid 6 (Example 6)

An aqueous solution containing tetrapropylammonium hydroxide (TPAH), as an organic alkali, at a concentration of 1 mol/L was prepared.

(7) Ruthenium Oxide Gas Absorbent Liquid 7 (Example 7) A 1 mol/L tetramethylammonium hydroxide aqueous solution containing 5% by mass of malonic acid was prepared.

(8) Ruthenium Oxide Gas Absorbent Liquid 8 (Example 8) A 1 mol/L tetramethylammonium hydroxide aqueous solution containing 5% by mass of citric acid was prepared.

(9) Ruthenium Oxide Gas Absorbent Liquid 9 (Example 9)

A 1 mol/L tetramethylammonium hydroxide aqueous solution containing 5% by mass of citric acid and 5% by mass of tetrapropylammonium chloride was prepared.

(10) Ruthenium Oxide Gas Absorbent Liquid 10

### Comparative Example 1

As an aqueous alkali solution, a 1 mol/L sodium hydroxide aqueous solution was prepared.

### Examples 1 to 9 and Comparative Example 1

Experimental Procedure:

(1) 10 ml of 2 mass % sodium hypochlorite aqueous solution was placed in an 85-ml glass sealed vessel. Next, the pH was adjusted with sodium hydroxide to 12. Then, a silicon wafer on which ruthenium had been sputtered and deposited (5×5 mm; Ru film thickness: 20 nm; Ru amount: 5.4×10⁻⁸ mol) was immersed therein at 23° C. for 15 min. Note that in Examples 4 to 9, the Ru film thickness was 5 nm (Ru amount: 1.35×10⁻⁸ mol).

Whether all Ru on the wafer had been dissolved was checked by Ru film thickness measurement using XRF.

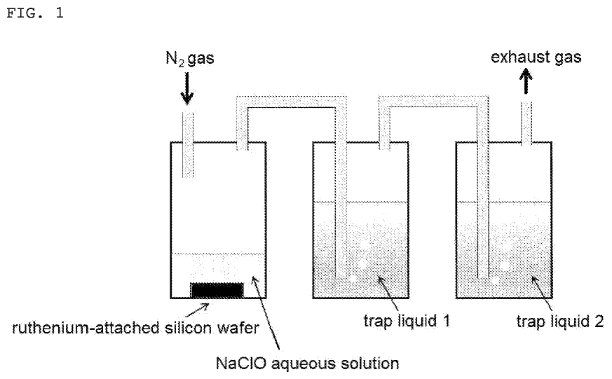

(2) After that, nitrogen gas was made to flow in the sealed vessel at 300 ml/min for 15 min. A ruthenium oxide gas generated while the ruthenium-attached silicon wafer was immersed was absorbed sequentially in gas trap liquid 1 and gas trap liquid 2 as illustrated in the schematic diagram of FIG. 1.

The same ruthenium oxide gas absorbent liquid was used as the gas trap liquids 1 and 2. Then, the volume of each absorbent liquid was set as in Table 1 and the above-described ruthenium oxide gas absorbent liquids 1 to 10 were each used for evaluation.

(3) 10 ml of each of the gas trap liquid 1 or the gas trap liquid 2 was sampled. Then, 20 ml of hydrochloric acid and ultrapure water were added to have a total volume of 100 ml. The resulting mixture was allowed to stand for 24 h. They were each used as a measurement liquid.

(4) The measurement liquid was measured by ICP-OES (iCAP 6500 Duo, manufactured by Thermo Fisher Scientific Inc.; measurement wavelength: 240.2 nm), and Ru was quantified.

(5) The measurement liquid was also measured by ICP-MS (ICP-MS 7900, manufactured by Agilent Technologies, Inc.; Ru detection m/z=101), and Ru was quantified.

(6) Ru was not detected in the gas trap liquid 2 when any of the ICP-OES or ICP-MS measurement method was used. Thus, the amount of Ru absorbed in the gas trap liquid 1 was determined as the quantified value for ruthenium oxide.

Note that in Examples 1 to 9 and Comparative Example 1, measurement was repeated 10 times under the same conditions using the gas trap liquid 2, and the standard deviation (a) of the measured values was calculated. The lower limit of quantification is the lowest amount that can be quantified as an analytical value, and was set to an amount 10 times a. Table 1 collectively shows the results.

TABLE 1

| | | ruthenium | | | | lower limit | |
|---|---|---|---|---|---|---|---|
| | Concen- | oxide gas | | | | of quantification | |
| | tration | absorbent liquid | | quantified value for ruthenium oxide | | of ruthenium | |
| | of hydroxide | | volume of | (Ru-amount in gas trap liquid 1) (mol) | | oxide (mol) | |
| | ion [mol/l] | No. | liquid(ml) | ICP-OES | ICP-MS | ICP-OES | ICP-MS |
| Example1 | 1.0 | 1 | 20 | below the lower limit of | 3.6 × 10⁻¹¹ | 1.4 × 10⁻⁸ | 1.0 × 10⁻¹¹ |
| | | | | quantification | | | |
| Example 2 | 1.0 × 10⁻³ | 2 | 20 | below the lower limit of | 3.6 × 10⁻¹¹ | 1.4 × 10⁻⁸ | 1.0 × 10⁻¹¹ |
| | | | | quantification | | | |
| Example 3 | 2.0 | 3 | 20 | below the lower limit of | 3.6 × 10⁻¹¹ | 1.4 × 10⁻⁸ | 1.0 × 10⁻¹¹ |
| | | | | quantification | | | |
| Example 4 | 1.0 | 4 | 20 | below the lower limit | below the lower | 1.4 × 10⁻⁸ | 1.0 × 10⁻¹¹ |
| | | | | of quantification | limit of | | |
| | | | | | quantification | | |
| Example 5 | 1.0 | 5 | 15 | below the lower limit of | 0.9 × 10⁻¹¹ | 1.05 × 10⁻⁸ | 0.75 × 10⁻¹¹ |
| | | | | quantification | | | |
| Example 6 | 1.0 | 6 | 10 | below the lower limit of | 0.9 × 10⁻¹¹ | 0.7 × 10⁻⁸ | 0.5 × 10⁻¹¹ |
| | | | | quantification | | | |
| Example 7 | 1.0 | 7 | 15 | below the lower limit of | 0.9 × 10⁻¹¹ | 1.05 × 10⁻⁸ | 0.75 × 10⁻¹¹ |
| | | | | quantification | | | |
| Example 8 | 1.0 | 8 | 15 | below the lower limit of | 0.9 × 10⁻¹¹ | 1.05 × 10⁻⁸ | 0.75 × 10⁻¹¹ |
| | | | | quantification | | | |
| Example 9 | 1.0 | 9 | 10 | below the lower limit of | 0.9 × 10⁻¹¹ | 0.7 × 10⁻⁸ | 0.5 × 10⁻¹¹ |
| | | | | quantification | | | |
| Comparative | 1.0 | 10 | 20 | below the lower limit of | — | 1.4 × 10⁻⁸ | — |
| Example 1 | | | | quantification | | | |

Since a large amount of sodium was contained in Comparative Example 1, the evaluation by ICP-MS was impossible. The lower limit of quantification by ICP-OES was 1.4×10⁻⁸ mol.

Examples 1 to 3 have demonstrated that ruthenium oxide contained in each absorbent liquid can be quantified with high sensitivity even when the concentration of hydroxide ions contained in the absorbent liquid is transformed.

In Example 4, in which the ruthenium film thickness is halved as compared with Example 1, the concentration of ruthenium contained in the absorbent liquid decreased, and the value for ruthenium measured by ICP-MS was below the lower limit of quantification.

In Examples 5 and 6, in which each absorbent liquid contained a tetrapropylammonium ion having a ruthenium trapping effect, the volume of the absorbent liquid was able to be reduced. Further, in Example 7 or 8, in which malonic acid or citric acid which exerts a ruthenium trapping effect, was contained as a ligand in each absorbent liquid, the volume of the absorbent liquid was able to be reduced.

In Example 9, in which in addition to the ligand, an onium salt other than the hydroxide was added to the absorbent liquid, the volume of the absorbent liquid was further able to be reduced as compared with Examples 7 and 8.

As above mentioned, in Examples 5 to 9, it is possible to lower the lower limit of quantification of ruthenium oxide when compared with Example 4, therefore, ruthenium oxide was successfully quantified with high sensitivity by ICP-MS.

In view of the above results, the present invention made it possible to increase 1000-fold the sensitivity for the quantification of ruthenium oxide. Also, the liquid volume of the absorbent liquid can be reduced to markedly increase the sensitivity without an operation such as concentration. This was effective in the case of any of ICP-MS or ICP-OES.

1. A ruthenium oxide gas absorbent liquid comprising an organic alkali solution containing an onium salt composed of an onium ion and an anion, at least part of which is a hydroxide ion, wherein the hydroxide ion has a concentration ranging from more than 1×10⁻⁷ mol/L to 6 mol/L or less.

2. The ruthenium oxide gas absorbent liquid according to claim 1, wherein the onium ion is a quaternary onium ion represented by formula (1) or a tertiary onium ion represented by formula (2): 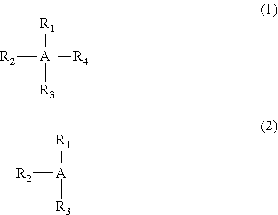
wherein in formula (1), A⁺ is an ammonium ion or a phosphonium ion, and R₁, R₂, R₃, and R₄ are each a C₁₋₂₅ alkyl group, an allyl group, a C₁₋₂₅ alkyl-containing aralkyl group, or an aryl group, provided that when R₁, R₂, R₃, or R₄ is an alkyl group or an allyl group, at least one hydrogen of R₁, R₂, R₃, or R₄ is optionally substituted by a hydroxyl group, a carboxyl group, a cyano group, an amino group, a thiol group, a halogen group, or a sulfonic acid group and at least one hydrogen of a ring of the aryl group or an aryl group in the aralkyl group is optionally substituted by a fluorine atom, a chlorine atom, a C₁₋₁₀ alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom; and in formula (2), A⁺ is a sulfonium ion, and R₁, R₂, and R₃ are each a C₁₋₂₅ alkyl group, an allyl group, a C₁₋₂₅ alkyl-containing aralkyl group, or an aryl group, and at least one hydrogen of a ring of the aryl group or an aryl group in the aralkyl group is optionally substituted by a fluorine atom, a chlorine atom, a C₁₋₁₀ alkyl group, a C₂₋₁₀ alkenyl group, a C₁₋₉ alkoxy group, or a C₂₋₉ alkenyloxy group, where at least one hydrogen is optionally substituted by a fluorine atom or a chlorine atom.

3. The ruthenium oxide gas absorbent liquid according to claim 2, wherein the onium ion is represented by formula (1), and is a quaternary onium ion where R₁, R₂, R₃, and R₄ are each a C₁₋₄ alkyl group.

4. The ruthenium oxide gas absorbent liquid according to claim 2, wherein the quaternary onium ion is an ammonium ion.

5. The ruthenium oxide gas absorbent liquid according to claim 4, wherein the ammonium ion is a tetraalkylammonium ion.

6. The ruthenium oxide gas absorbent liquid according to claim 5, wherein the tetraalkylammonium ion is at least one tetraalkylammonium ion selected from a tetramethylammonium ion, a tetraethylammonium ion, a tetrapropylammonium ion, or a tetrabutylammonium ion.

7. The ruthenium oxide gas absorbent liquid according to claim 1, further comprising, as a ruthenium oxide gas generation inhibitor, an onium salt composed of an onium ion and another anion other than a hydroxide.

8. The ruthenium oxide gas absorbent liquid according to claim 7, wherein the other anion is at least one ion selected from a fluoride ion, a chloride ion, an iodide ion, a nitrate ion, a phosphate ion, a sulfate ion, a hydrogen sulfate ion, a methane sulfate ion, a perchlorate ion, a chlorate ion, a chlorite ion, a hypochlorite ion, an orthoperiodate ion, a metaperiodate ion, an iodate ion, an iodite ion, a hypoiodite ion, an acetate ion, a carbonate ion, a bicarbonate ion, a fluoroborate ion, or a trifluoroacetate ion.

9. The ruthenium oxide gas absorbent liquid according to claim 1, further comprising, as a ruthenium oxide gas generation inhibitor, a ligand coordinated to ruthenium.

10. The ruthenium oxide gas absorbent liquid according to claim 9, wherein the ligand coordinated to ruthenium is oxalic acid, dimethyl oxalate, 1,2,3,4,5,6-cyclohexanecarboxylic acid, succinic acid, acetic acid, butane-1,2,3,4-tetracarboxylic acid, dimethylmalonic acid, glutaric acid, di-glycolic acid, citric acid, malonic acid, 1,3-adamantane dicarboxylic acid, or 2,2-bis(hydroxymethyl)propionic acid.

11. The ruthenium oxide gas absorbent liquid according to claim 1, wherein the onium ion has a concentration of from 1×10⁻⁷ mol/L to 8 mol/L.

12. The ruthenium oxide gas absorbent liquid according to claim 1, further comprising water or an organic solvent.

13. The ruthenium oxide gas absorbent liquid according to claim 12, wherein the organic solvent has a relative permittivity of 45 or less.

14. The ruthenium oxide gas absorbent liquid according to claim 12, wherein the organic solvent is at least one compound selected from the group consisting of sulfolanes, alkylnitriles, halogenated alkanes, ethers, esters, aldehydes, ketones, and alcohols.

15. The ruthenium oxide gas absorbent liquid according to claim 12, wherein the organic solvent in a treatment liquid has a concentration of 0.1 mass % or more.

16. An analysis method for ruthenium oxide in a process gas, the method comprising: bringing a ruthenium oxide gas-containing process gas into contact with the ruthenium oxide gas absorbent liquid according to claim 1 to recover the ruthenium oxide gas from the process gas; and then analyzing an amount of ruthenium oxide in the ruthenium oxide gas absorbent liquid.

17. The analysis method according to claim 16, wherein the process gas is derived from a semiconductor-use chemical liquid used for processing a ruthenium metal-containing semiconductor material.

18. A trap device including a trap unit disposed in an exhaust path during processing a ruthenium metal-containing semiconductor device and configured to recover a ruthenium oxide component in an exhaust gas,
the trap unit including: a container filled with the ruthenium oxide gas absorbent liquid according to claim 1; a supply unit; and a drainage pipe for discharging the ruthenium oxide gas absorbent liquid after absorption.

19. A ruthenium oxide gas quantitative analyzer including:
a trap device including a trap unit disposed in an exhaust path during processing a ruthenium metal-containing semiconductor device and configured to recover a ruthenium oxide component in an exhaust gas,
the trap unit including: a container filled with the ruthenium oxide gas absorbent liquid according to claim 1, a supply unit, and a drainage pipe for discharging the ruthenium oxide gas absorbent liquid after absorption; and an analysis device for quantifying the ruthenium oxide component in the absorbent liquid sampled from the trap device.

20. The quantitative analyzer according to claim 19, wherein the analysis device is ICP emission spectrometry, ICP mass spectrometry, or ultraviolet-visible spectroscopy (UV-VIS).
